(19) United States
(12) Patent Application Publication
(10) Pub. No.: US 2022/0168003 A1
(43) Pub. Date: Jun. 2, 2022

(54) DILATION AND STONE RETRIEVAL CATHETER

(71) Applicant: OLYMPUS AMERICA INC., Center Valley, PA (US)

(72) Inventor: Peter J. Crowley, Norfolk, MA (US)

(21) Appl. No.: 17/539,531

(22) Filed: Dec. 1, 2021

Related U.S. Application Data
(60) Provisional application No. 63/119,781, filed on Dec. 1, 2020.

(51) Int. Cl. A61B 17/221; A61M 29/02

(52) U.S. Cl. CPC A61B 17/221; A61B 2017/22061; A61M 29/02

(57) ABSTRACT

A medical device for capturing renal calculi includes an inflatable balloon, an elongated shaft extending through an orifiace defined by the balloon and attached thereto, a basket apparatus deployable via a distal end of the shaft, and a sheath. The basket includes two or more wire loops that capture calculi and have a collapsed configuration when the basket is positioned within the distal end of the shaft. The medical device can be used by dilating a percutaneous tract by inflating the balloon. A sheath can be advanced over the balloon to create an access route. The basket can be utilized without the removal of the balloon. The balloon can be deflated to allow the shaft and basket to be manipulated in the access route. The shaft and basket can be retracted from the access route to retrieve renal calculi.

### CROSS-REFERENCE TO RELATED APPLICATION

This application claims the benefit of priority to U.S. Provisional Patent Application Ser. No. 63/119,781, filed Dec. 1, 2021, the contents of which are hereby incorporated by reference in their entirety.

### TECHNICAL FIELD

This document pertains generally, but not by way of limitation, to devices and methods for percutaneous nephrolithotomy.

### BACKGROUND

A minimally invasive surgical device can provide access to surgical sites in a human body through a body opening, cavity, or tract. In certain urological procedures, such as percutaneous nephrolithotomy (PCNL), a probe is inserted to run from the surface of a patient's skin to the target surgical site. The probe, which can be a needle, can create a passage such that a guide wire can be threaded from the surface of the skin to the surgical site. Later in the procedure, the initial insertion can be dilated to accommodate one of several other medical devices.

### SUMMARY

In an approach to PCNL, a high-pressure balloon catheter can be used such as to dilate the initial insertion. The balloon can be advanced over the safety guide wire and inflated with a dilute contrast media solution, to enlarge the tract. Once the balloon is inflated to a suitable diameter, a sheath can be advanced over the balloon. The balloon can then be deflated and removed from the sheath such as to provide access route for other medical devices. For removal of a calculus from a kidney, a basket retriever can be inserted through the access route and maneuvered to grasp the calculus and remove it from the patient. The present inventors have recognized, among other things, that the need for entry and removal of several devices can slow the procedure and can necessitate more than one caregiver to assist in operation.

Aspect 1 can include or use a combination access and removal device for use in percutaneous nephrolithotomy such as to remove a kidney stone from a patient, such as can include or use an inflatable balloon configured such as to dilate a percutaneous nephrolithotomy access route from an external location on the patient to a location within a kidney of the patient, an elongated shaft extending through a central lumen defined by the balloon and attached thereto, a basket apparatus deployable via a distal end of the shaft and configured such as to capture a kidney stone or fragment or both, and a sheath configured such as to be extended over the inflatable balloon such as to provide a port extending along the percutaneous nephrolithotomy access route such as to provide secondary instrument access to the kidney including when the shaft is located within the port with the balloon in a deflated state. Aspect 2 can include or use, or be optionally combined with the subject matter of Aspect 1, to optionally include or use a basket that can be configured such as to be retrieved through the port via retraction. Aspect 3 can include or use, or can optionally be combined with the subject matter of one of any combination of Aspects 1 or 2 to optionally include or use a balloon and basket that can be attached to the same shaft. Aspect 4 can include or use, or can optionally be combined with the subject matter of one of any combination of Aspects 1 through 3 to optionally include or use the that device can be configured such as to allow for entry of the basket through a distal end of the port without removal of the balloon. Aspect 5 can include or use, or can optionally be combined with the subject matter of one of any combination of Aspects 1 through 4 to optionally include or use a basket that can be configured such as to be collapsible within the distal end of the shaft. Aspect 6 can include or use, or can optionally be combined with the subject matter of one of any combination of Aspects 1 through 5 to optionally include or use a basket actuator located at the proximal end of the shaft, the basket actuator being configured such as to deploy the basket apparatus when the shaft is located within the port. Aspect 7 can include or use, or can optionally be combined with the subject matter of one of any combination of Aspects 1 through 6 to optionally include or use a basket that can be configured such as to be capable of withstanding lithotripsy via the secondary instrument and remain safely removable. Aspect 8 can include or use, or can optionally be combined with the subject matter of one of any combination of Aspects 1 through 7 to optionally include or use a basket that can be configured such as to break non-traumatically when struck by the secondary instrument. Aspect 9 can include or use, or can optionally be combined with the subject matter of one of any combination of Aspects 1 through 8 to optionally include or use a balloon that can have a length of at least 12 centimeters. Aspect 10 can include or use, or can optionally be combined with the subject matter of one of any combination of Aspects 1 through 9 to optionally include or use a balloon that can have a length of less than 14 centimeters. Aspect 11 can include or use, or can optionally be combined with the subject matter of one of any combination of Aspects 1 through 10 to optionally include or use a balloon that when in an inflated state can have an outer diameter of at least 10 millimeters. Aspect 12 can include or use, or can optionally be combined with the subject matter of one of any combination of Aspects 1 through 11 to optionally include or use a balloon that when in an inflated state can have an outer diameter of less than 12 millimeters. Aspect 13 can include or use, or can optionally be combined with the subject matter of one of any combination of Aspects 1 through 12 to optionally include or use a shaft that can include or use a longitudinal lumen and can further comprise a basket manipulation member configured such as to extend through the lumen of the shaft and can connect to the basket at a distal end of the basket manipulation member, such as to permit removal of a kidney stone, fragment, or both via the lumen of the shaft. Aspect 14 can include or use, or can optionally be combined with the subject matter of one of any combination of Aspects 1 through 13 to optionally include or use a basket manipulation member that can be configured such as to provide a first position wherein the basket is near the distal end of the shaft, and a second position wherein the basket extends outward from the distal end of the shaft. Aspect 15 can include or use, or can optionally be combined with the subject matter of one of any combination of Aspects 1 through 14 to optionally include or use a device that can include or use an extension actuator located at the proximal end of the shaft and the extension actuator can be configured such as to extend the move the basket manipulation member from the first position to the second position.

Aspect 16 can include or use, or can optionally be combined with the subject matter of one of any combination of Aspects 1 through 15 to optionally include or use a method for performing percutaneous nephrolithotomy such as to remove a kidney stone such as can include or use dilating, using an inflatable balloon on an elongated shaft, a percutaneous nephrolithotomy access route from an external location on the patient to a location within a kidney of the patient, providing a port, such as by extending a sheath over the inflatable balloon, along the percutaneous nephrolithotomy access route from the external location on the patient to the location within the kidney of the patient, and capturing, using a basket deployed via a distal end of the shaft, a kidney stone or fragment, or both. Aspect 17 can include or use, or can optionally be combined with the subject matter of one of any combination of Aspects 1 through 16 to optionally include or use retrieving a captured stone or fragment or both by retracting the basket through the port. Aspect 18 can include or use, or can optionally be combined with the subject matter of one of any combination of Aspects 1 through 17 to optionally include or use deflating the balloon when the port has been provided. Aspect 19 can include or use, or can optionally be combined with the subject matter of one of any combination of Aspects 1 through 18 to optionally include or use deflating the balloon which can permit concurrent access of the shaft and a secondary instrument to the port. Aspect 20 can include or use, or can optionally be combined with the subject matter of one of any combination of Aspects 1 through 19 to optionally include or use dilating which can include or use inflating the balloon with a non-gaseous medium to a pressure of at least 18 atmospheres. Each of these non-limiting examples can stand on its own or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals can describe similar components in different views. Like numerals having different letter suffixes may represent different instances of similar components. The drawings illustrate generally, by way of example, but not by way of limitation, various embodiments discussed in the present document.

FIG. 1 is a perspective view of an example of a PCNL device.

FIG. 2 is a side view of an example of a PCNL device in operation.

FIG. 3A is a side view of a deflated balloon attached to a shaft of an example of a PCNL device.

FIG. 3B is a side view of an inflated balloon attached to a shaft of an example of a PCNL device.

FIG. 4A is a perspective view of a sheath partially extended over an inflated balloon of an example of a PCNL device.

FIG. 4B is a perspective view of a sheath extended over an inflated balloon of an example of a PCNL device.

FIG. 4C is a perspective view of a sheath extended over a deflated balloon of an example of a PCNL device.

FIG. 5A is a perspective view of an undeployed basket apparatus of an example of a PCNL device.

FIG. 5B is a perspective view of a deployed basket apparatus of an example of a PCNL device.

FIG. 5C is a side view of an example of a PCNL device and a controller.

FIG. 6A is a perspective view of a basket apparatus of an example of a PCNL device.

FIG. 6B is a side view of an example of a PCNL device and a controller.

FIG. 7 is a side view of an example of a PCNL device in operation.

FIG. 8A is a side view of an example of a PCNL device in operation.

FIG. 8B is a side view of an example of a PCNL device in operation.

### DETAILED DESCRIPTION

The present disclosure, in one or more examples, relates to devices and methods for percutaneous nephrolithotomy (PCNL). More particularly, the present disclosure relates to a PCNL dilation and calculus retrieval device and the method for safe dilation and retrieval. A PCNL procedure can be undergone to remove a calculus from a kidney through a small puncture in a patient's skin. A small incision can be made in a patient's skin, and a PCNL needle can be passed through a kidney calyx and into a renal pelvis of a kidney. The needle can be positioned by one of several imaging techniques such as fluoroscopy or endoscopy. A guide wire, referred to herein as the safety guide wire, can be passed through the needle and introduced to the renal pelvis, and the needle can be removed, leaving the safety guide wire behind. In an example, the safety guide wire can be anchored at or near the ureter or within a kidney calyx and can be used, e.g., as a failsafe against closure of the puncture resulting in loss of access to the renal pelvis during the procedure.

One of several medical devices can be passed over the safety guide wire to dilate the tract to the renal pelvis. One approach is to insert a high-pressure balloon catheter over the safety guide wire and to inflate the balloon with a dilute contrast media solution. Inflation of the balloon can dilate the tract, and a rigid sheath can be advanced over the balloon. Advancing the sheath can involve applying axial force to the sheath along with rotation of the sheath. Once the sheath is fully advanced, the balloon can be deflated and withdrawn from the sheath to create an access port to the renal pelvis. Once the balloon catheter is withdrawn, a professional can insert one of several medical devices into the access port to facilitate calculus removal. A problem with that approach, however, is that the balloon must be withdrawn before access to the port can be utilized by a removal device. Further, multiple devices must be handled simultaneously and swapped during the procedure. This takes extra time and creates a more costly procedure due to the need for multiple devices. The present devices and techniques can help avoid such problems because of the lack of need to remove the balloon catheter to use a removal device through the port.

The present disclosure relates to, among other things, a device and a method enabling dilation of a percutaneous tract and safe calculus removal without the need for removing the dilating device to gain access to the renal pelvis. The device also can help reduce the number of exchanges of medical devices through the access port and can help enable a procedure requiring fewer professionals. The device can be configured to be passed over a safety guide wire percutaneously to reach a renal pelvis of a kidney. Additionally, the device can be configured to dilate a percutaneous tract with a balloon catheter and to create an access port to the renal pelvis. The device can be configured to deploy a basket apparatus from the distal end of the balloon catheter and to capture a renal calculus therewith. For example, the basket apparatus can extend from the balloon catheter via a basket manipulation member. The device can be configured to safely remove a target object such as a calculus from the kidney.

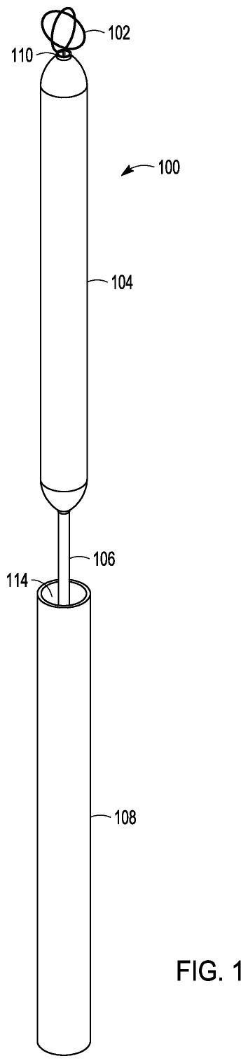

FIG. 1 shows a perspective view of an example of a PCNL device. A PCNL device 100 can include a balloon 104, a shaft 106, a basket apparatus 102, and a sheath 108. The shaft 106 can extend through the balloon 104. The balloon 104 can be attached to the shaft 106 by a variety of methods. The basket apparatus 102 can be deployable from the distal end 110 of the shaft 106. The outer diameter of the balloon, 104, when inflated, can be approximately equivalent to the inner diameter of the sheath 108, such that the inflated balloon can slidingly contact with an inner surface 114 of the sheath 108. The inner surface 114 of the sheath 108 can include a lubricious coating such as to help the sliding of the sheath 108 over the inflated balloon 104.

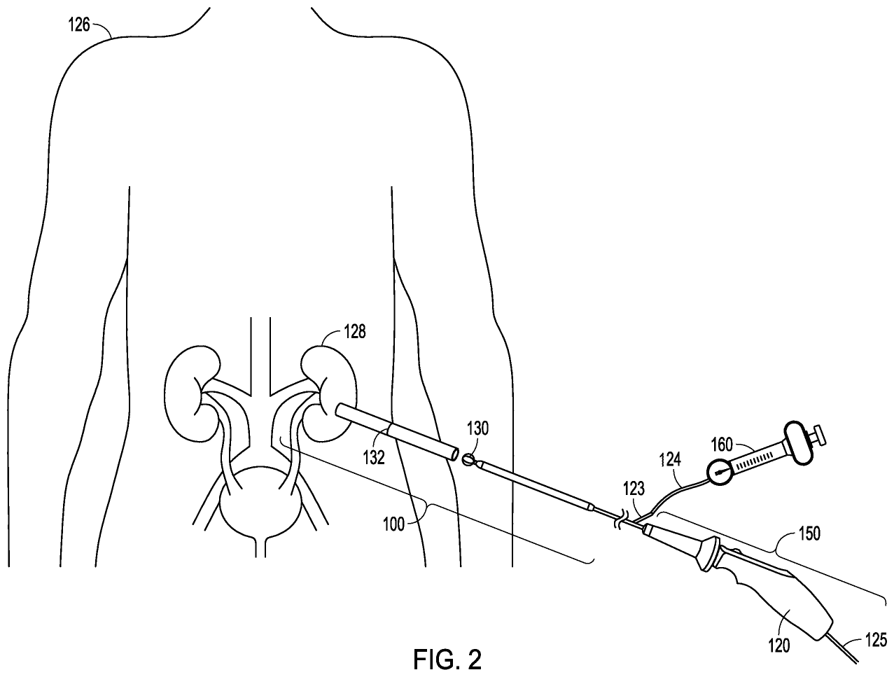

FIG. 2 shows a side view of an example of a PCNL device in operation. FIG. 2 shows an example of the device 100 in a procedure of a patient body 126, having extracted a calculus 130. As referred to herein, a calculus 130 such as a kidney stone can be a single calculus, calculi, calculi fragments, or other foreign bodies which can exist in a kidney 128. The device 100 can extend through an access route 132 to access a kidney 128. The device 100 can be coupled to a controller 150. The controller can have a handle 120, a service line 125, and an inflation port 123. Alternatively, the inflation port 123 can be located on the proximal end 116 of the shaft 106, as depicted in FIG. 2. The handle 120 can be held by a professional and can allow for positioning of the device 100 relative to the patient body 126. An inflation line 124 can facilitate travel of fluid between the device 100 and a pump 160 via the inflation port 123. The service line 125 can be alternatively or additionally used to facilitate travel of the fluid. The service line 125 can also be used to supply power or pressurized gas to the device 100 and can be used to electrically connect the device 100 to a medical system. The service line 125 can also facilitate the supply or withdrawal of fluids from the device 100.

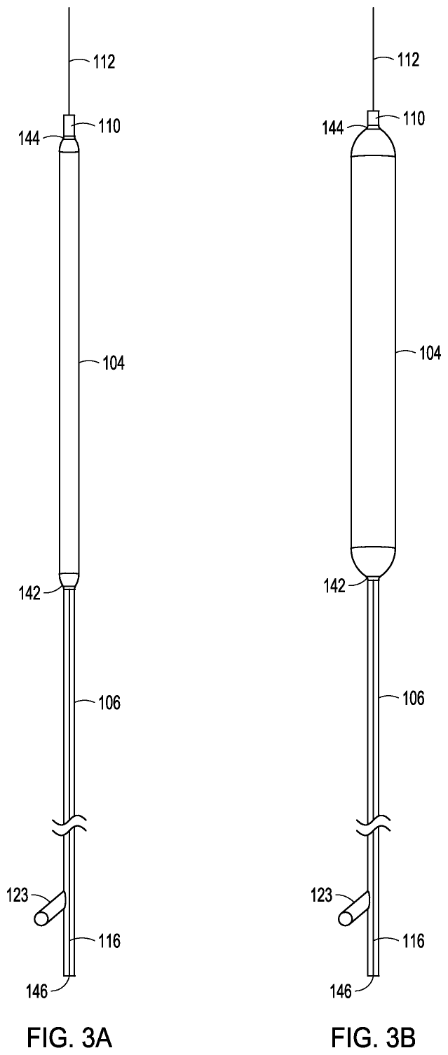

FIG. 3A shows a side view of a deflated balloon attached to a shaft of a PCNL device. The balloon 104 can be attached to the shaft 106 by a variety of methods, including by inserting the distal end 110 of the shaft 106 through a proximal mouth 142 and distal mouth 144 of the balloon 104 and bonding thereto, such as with an ultraviolet-curable adhesive. Alternatively or additionally, the balloon 104 can be bonded to the shaft 106 such as with the use of radiofrequency welding, solvent, or glue. Other suitable methods of attachment can also be used. The balloon can be attached to the shaft such as to locate the distal mouth 144 of the balloon 104 near the distal end 110 of the shaft 106. The shaft 106 can include or contain a fluid supply lumen 146 such as for supplying fluid from a proximal end 116 of the shaft 106 through the proximal mouth 142 of the balloon 104 and into the balloon 104, such as for inflating the balloon. The fluid can enter the supply lumen 146 via the inflation port 123. Fluid can be retracted from the balloon 104 through the fluid supply lumen 146 such as to deflate the balloon. The shaft 106 can be configured to be advanced over a safety guide wire 112 such as for insertion into an access route 132.

FIG. 3B shows a side view of an example of an inflated balloon attached to a shaft of a PCNL device. The balloon 104 can include or be formed of a material that can be substantially inelastic such as to stretch a relatively small amount (e.g., increasing in diameter within a range of about 5% to about 12%) under inflation pressures of 15 atmospheres or more. Various substantially non-compliant and non-porous elastomeric materials can be used, such as Nylon (Nylon 12), polyether block amide (PEBAX), PEBAX 4033, PEBAX 5533, PEBAX 6333, and poly(ethylene terephthalate) (PET). Other suitable materials can also be used. Typically, the balloon can have a burst pressure of at least 18 atm; and more preferably at least 20 atm, and most preferably as high as or higher than 30 atm. The balloon 104 can be configured to be inflated to a single predetermined or specific outer balloon diameter. The inflated outer balloon diameter can fall within a range from about 2 millimeters to about 30 millimeters. More preferably, the inflated balloon can fall within a range from about 8 millimeters to about 12 millimeters. The inflated outer balloon diameter can fall within any range of diameters sufficient to dilate the access route 132 for insertion of the sheath 108. Although the above balloon can be configured to be inflated to a single predetermined or specific outer balloon diameter, the inflated outer balloon diameter can be slightly larger or slightly smaller than the single predetermined or specific diameter of the balloon 104. The length of the balloon 104, extending from the proximal mouth 142 to the distal mouth 144 can be in a range from about 2 centimeters to about 25 centimeters. More preferably, the length of the inflated balloon 104 can be in a range from 12 centimeters to about 14 centimeters. The inflated balloon length can fall with any range of lengths sufficient to dilate along the length of the access route 132 for insertion of the sheath 108.

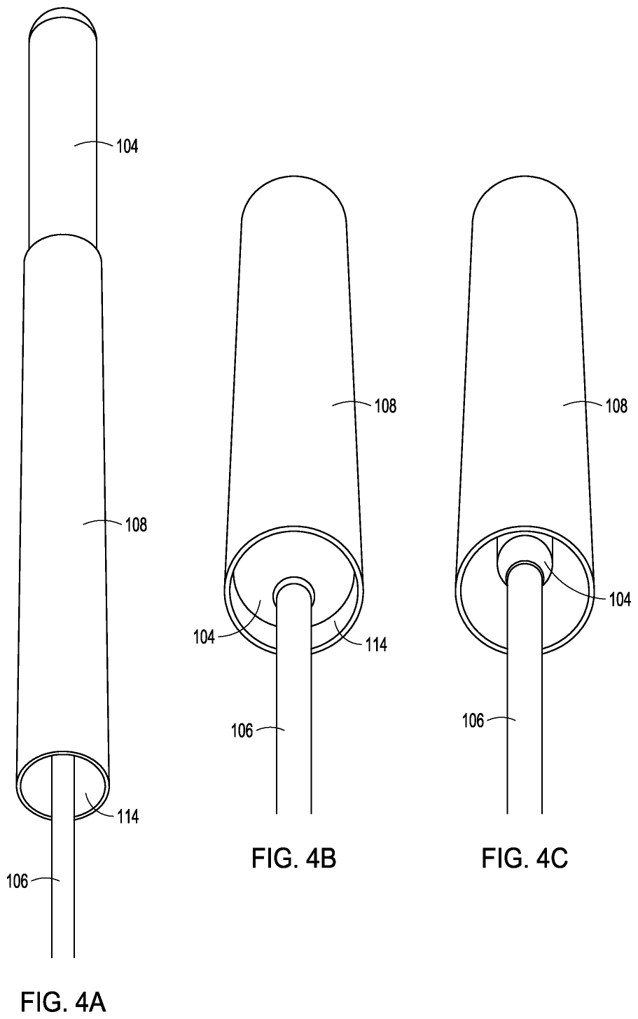

FIG. 4A shows a perspective view of a sheath partially extended over an example of an inflated balloon of a PCNL device. The sheath 108 can be shaped cylindrically and can be hollow such as to help establish a sheath lumen. The sheath 108 can be sized and shaped to be extended over the inflated balloon 104, the balloon 104 slidingly connecting to the inner surface 114 of the sheath 108 and articulating through the sheath lumen. The inner diameter of the sheath 108 and the outer diameter of the inflated balloon 104 can each be sized such as to reduce friction during sheath placement. The shaft 106 be elongated and can extend proximally out the proximal end of the sheath 108.

FIG. 4B shows a perspective view of an example of a sheath extended over an inflated balloon of a PCNL device. The sheath 108 can be advanced over the inflated balloon 104 such that the sheath substantially encloses the balloon 104. The sheath 108 can be sized at a length near or approximately equal to the length of the balloon 104.

FIG. 4C shows a perspective view of an example of a sheath extended over a deflated balloon of a PCNL device. The sheath 108 can remain extended over the balloon 104 while the balloon 104 is deflated. The sheath 108 can be constructed of Polytetrafluoroethylene (PTFE) or other materials suitable for medical use. The balloon 104 can be deflated by depressurizing or draining the fluid back through the fluid supply lumen 146, such as by opening a valve or decreasing or deactivating a pressure source. Deflation of the balloon 104 can allow for flexible movement of the shaft 106 within the sheath 108. Deflation of the balloon can also allow for the insertion of a secondary instrument 136, such as a laser fiber or a lithotripter, through the sheath 108 defining an access port without the need to remove the shaft 106 and the balloon 104 from the sheath 108. In another example, an access port can be defined without insertion of the sheath 108. The balloon 104 can instead be removably attached to the shaft 106. Instead of advancing a sheath over the balloon to define an access port, an inflated balloon 104 can include a balloon lumen. The balloon 104 can be sized and shaped to allow the shaft 106 to be slidingly connected to the balloon lumen and to allow for the shaft 106 and the deployed basket apparatus 102 to be retrieved through the proximal end of the balloon lumen.

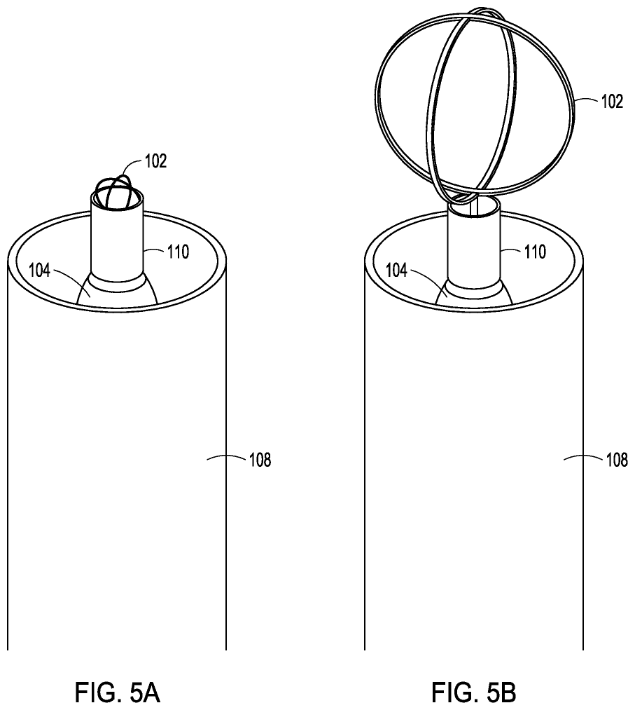

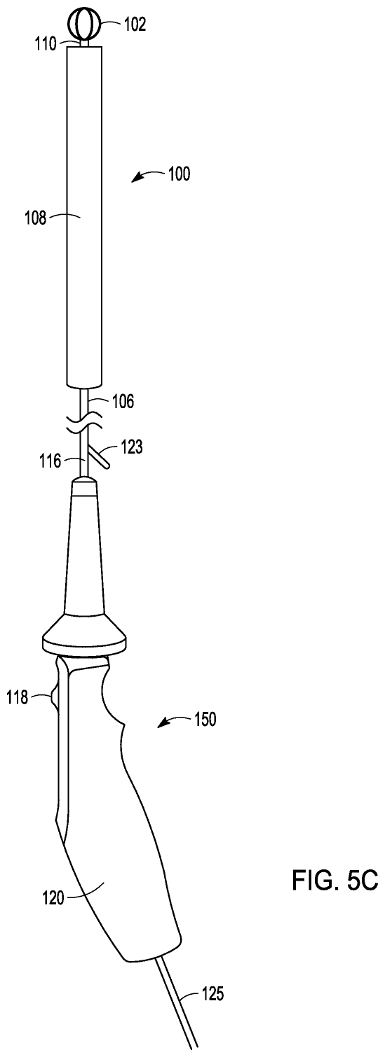

FIGS. 5A-5C depict perspective views and a side view of an example of a basket apparatus of a PCNL device. While exemplified herein as a basket apparatus 102, other retrieval devices can also be additionally or alternatively used at the distal end 110 of the shaft 106. The basket apparatus 102 can also be referred to herein as the basket 102. As shown in FIG. 5A, the basket 102 can be held undeployed from the distal end 110 of the shaft 106. In examples where the basket 102 is initially undeployed, the basket 102 can be initially collapsed within the distal end 110 of the shaft 106. Various mechanisms can be used to move the basket 102 such as to exit the distal end 110 of the shaft 106 and to expand therefrom, achieving a deployed position. A basket actuator 118 can deploy the basket 102 from the distal end 110 of the shaft 106, as shown in FIG. 5B. In one example, as depicted in FIG. 5C, the basket actuator 118 can be a button on a handle 120 of a controller 150. The button can function to deploy the basket 102 from the distal end 110 of the shaft 106 mechanically. An illustrative example of a mechanical basket actuator 118 is described in U.S. Pat. No. 10,349,961, which is incorporated by reference herein in its entirety, including for its teaching of basket actuation mechanism, which can be used in combination with the PCNL device described in the present disclosure. Alternatively or additionally, the button can function to deploy the basket 102 from the distal end 110 of the shaft 106 electrically or pneumatically. The button can be electrically coupled to a motor such as to move the basket 102 to the deployed position. The button can be pneumatically coupled to a pressurized gas cylinder such as to utilize a flow of pressurized gas from the cylinder to move the basket 102 to the deployed position. In another example, the handle 120 can have a mechanical trigger. The trigger can function such as to deploy the basket by moving the basket 102 from the distal end 110 of the shaft 106. The trigger can be operated by applying hand pressure to the trigger, squeezing the trigger, deflecting a basket manipulation member distally, and causing the basket to deploy from the distal end 110 of the shaft 106. Alternatively, the basket can be deployed manually Other mechanisms for actuation are commonly known by persons skilled in the art and can be used herewith to deploy the basket 102. In the above examples, the various mechanisms used to deploy the basket 102 can also function to move the basket 102 from the deployed position back to the undeployed position for subsequent use. Alternatively, the basket can be deployed manually. A rod can connect to the basket 102 and extend through a lumen of the shaft 106 and out the proximal end 116 of the shaft. Manual, distal movement of the rod at the proximal end 116 can actuate the basket 102, moving the basket 102 to the deployed position. Manual, proximal movement of the rod at the proximal end 116 can move the basket 102 from the deployed position back to the undeployed position for subsequent use. In some examples, the basket 102 can alternatively be fixed at the distal end 110 of the shaft 106 initially in the deployed position such as to allow for use of the deployed basket 102 without the need for mechanical, electrical, or manual actuation.

When the basket 102 is deployed and the balloon 104 deflated, movement of the shaft 106 at the proximal end 116 can control and place the basket 102. The shaft 106 can be sized and shaped relative to the sheath 108 such as to allow the shaft 106 to be manipulated within the sheath 108 at a wide range of angles and positions, allowing the professional to capture a calculus from a wide range of positions in the renal pelvis 134. As depicted in FIG. 5B, the basket 102 can be formed of a pair of looped wires. Although described herein as a pair of looped wires, a plurality of looped wires can also be used and can be oriented to similarly function as described below. Furthermore, the basket 102 can be similar configurations of wires having an odd number of lobes and can be oriented to similarly function as described below. The pair of looped wires can be oriented such that the mid region of the wires intersect at the distal most end of the basket 102. The ends of each of the wires can be attached to the distal end 110 of the shaft 106. The ends of each of the wires can be attached to a basket manipulation member. Alternatively or additionally, the ends of each of the wires can extend into and through the shaft 106 and can be attached to a winding spool mechanism. The pair of looped wires can be each attached to separate attachment points. One or both of the separate attachment points can be manipulated such as to orient the wires by rotation. The pair of looped wires can be biased or fixed such as to orient them orthogonally relative to each another and defining a capture configuration. The pair of looped wires can be manipulated such as to rotate the wires relative to one another such as to orient them non-orthogonally relative to each other and defining a release configuration. The pair of wires are able to be reset into a capture configuration after being moved to the release configuration. Each of the pair of looped wires can be shaped such that opposing sides of each respective loop follow straight, mirror paths as depicted in FIG. 5B. Alternatively, each of the pair of looped wires can be shaped such that opposing sides of each respective loop follow opposing helical paths or dual helical paths. An illustrative example of a basket with opposing sides of wire loops having helical paths is described in U.S. Pat. No. 7,101,379, which is incorporated by reference herein in its entirety, including for its teaching of helical wire loops, which can be used in combination with the PCNL device described in the present disclosure.

The basket 102 can be formed of wires that are preferably a superelastic shape-memory material. For instance, the wires of the basket 102 can be formed of Nitinol, a Ni—Ti alloy. Other alloys, such as Cu—Zn—Al, or Cu—Al—Ni can also be used. Alternatively, the wires of the basket 102 can be formed of another expandable metal, such as stainless steel, or of a synthetic material such as a plastic or other polymer. A basket 102 can be formed of looped wire and can be sized and shaped to capture a target object such as a calculus from the renal pelvis 134. The basket 102 can be shaped and formed with materials such that it can be struck with a secondary instrument and not fragment. The basket 102 can be shaped and formed with materials such that it can be subject to energy from a lithotripter, laser, or other secondary instrument 136 and not fragment. The basket 102 can also be shaped and formed with materials such that it can be struck with a secondary instrument or secondary instrument energy and still be completely retrieved safely from the renal pelvis 134. In an example, the basket 102 can be fixed at or near the distal end 110 of the shaft 106. The secondary instrument 136 can be used to help direct or manipulate the basket 102 towards a therapy location. In an example, the secondary instrument can include or use a superelastic or shape-memory material such as Nitinol, such as can provide an arc, when extended, such as which can be directed toward a target in part by rotating the secondary instrument 136 to sweep the extended arc portion, if desired. Here, the extended arc portion can be used such as to push the basket 102 towards the therapy location, or the extended arc portion can be used such as a contour which the basket 102 can follow towards the therapy location. In another example, the secondary instrument 136 can be another guide wire, referred to herein as the access guide wire. A distal end of the access guide wire can be positioned within the renal pelvis 134 at the therapy location, such as within one of the calyces, and can act such as a track or path for the basket 102 to follow. In an example, the basket 102 can contact the access guide wire and follow a contour thereof such as to help manipulate or guide the basket 102 to the therapy location. In another example, the basket 102 can be threaded by the access guide wire and the basket or an eyelet attached thereto can ride the wire to the therapy location.

Other examples can also be made using retrieval devices other than a basket, such as a grasper assembly, a jaw-type retrieval assembly, a scissors-type retrieval assembly, a vacuum tube, a suction catheter, a drainage tube, an irrigator, or other suitable retrieval device attached to or deploying from the distal end 110 of the shaft 106. While typically a basket can be the preferred retrieval device, the alternative retrieval devices described here can be utilized depending on the nature of the specific procedure. The alternative retrieval devices can be operated by manipulation of the handle 120 or the proximal end 116 of the shaft 106 in a similar fashion to that which has been described herein for the basket 102.

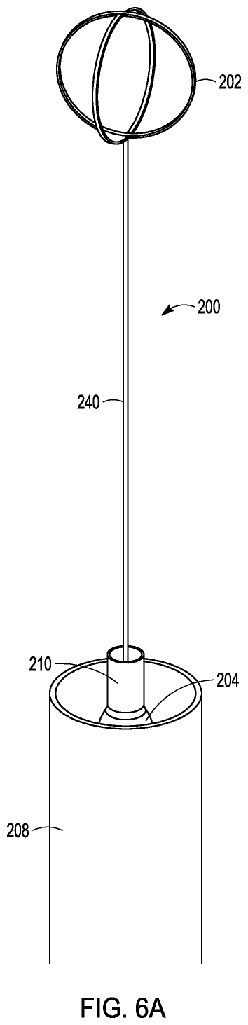

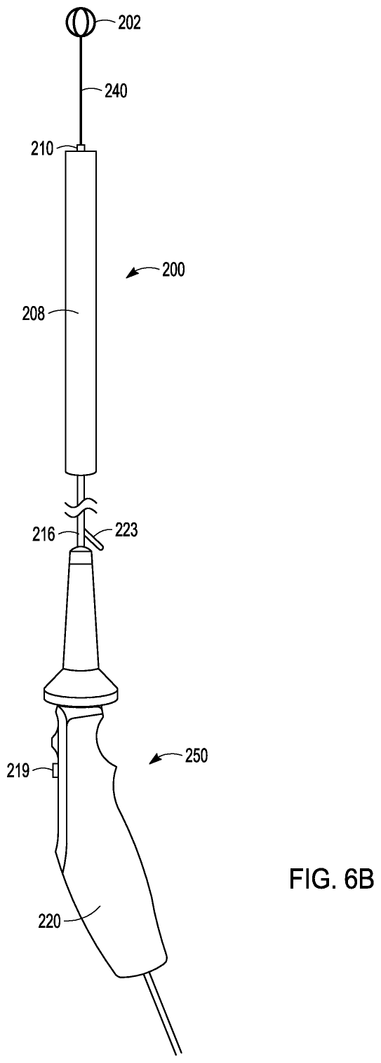

FIGS. 6A and 6B depict another example of a PCNL device. Device 200 is identical in most respects to device 100, but a basket manipulation member 240 and an extension actuator 219 have been added. The device 200 can include a basket 202 extendable from a distal end 210 of shaft by a basket manipulation member 240. In FIGS. 6A and 6B, the device 200, the basket manipulation member 240 can extend through a lumen at the distal end 210 of the shaft and connect to the basket 202 at a distal end of the basket manipulation member to permit removal of a calculus. The basket manipulation member 240 can be controllable via the extension actuator 219. In one example, the extension actuator 219 can be located on a handle 220 of a controller 250.

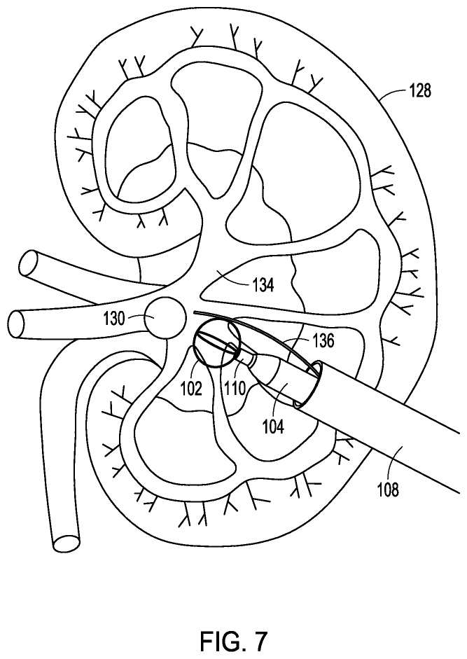

In operation and use, a device 100 of the present disclosure can help provide for safe removal of a calculus 130 from a renal pelvis 134 of a kidney 128 such as without the need to exchange a dilation device formed by a shaft 106 and a balloon 104 for a separate retrieval device. Instead, the device 100 contains a retrieval device such as a basket 102 on the distal end 110 of the shaft 106, such that the same device can be used for dilation and for calculus retrieval. In one example, a professional can insert a shaft 106 having a balloon 104 over a safety guide wire 112 that has been implanted through a percutaneous access route 132. The shaft 106 having the balloon 104 can be advanced through the access route 132 and into a renal pelvis 134 of a kidney. The access route 132 can be dilated by supplying fluid to the balloon 104 and inflating the balloon 104. The balloon 104 can be inflated by supplying fluid from a proximal end 116 of the shaft 106 through the proximal mouth 142 of the balloon 104 and into the balloon 104. In an example, the fluid can be a non-gaseous medium. The fluid can be a liquid such as a contrast media for diagnostic imaging as well as to provide a balloon inflation capability. The contrast media can be diluted with saline solution so that the combined fluid has a lower viscosity than the contrast media alone. The contrast media can be supplied by a fluid pump 160. The contrast media can also be supplied by any apparatus capable of supplying the contrast media to the device 100. In one example, the contrast media can be supplied to the device 100 via the controller 150 through the inflation line 124 into the inflation port 123. In another example, the contrast media can be supplied to the shaft 106 through the service line 125. The balloon 104 can be inflated to a specific or preselected pressure in a range from 2 atm to 30 atm. More preferably, the balloon 104 can be inflated to a specific or preselected pressure in a range from 15 atm to 20 atm. The balloon 104 can be inflated to a specific or preselected pressure in any range of pressures sufficient to dilate the access route 132 for insertion of the sheath 108. A rigid sheath 108 can be advanced over the balloon 104 and into the access route 132 to form an access port to the renal pelvis 134. Advancing the sheath 108 can involve applying axial force to the sheath 108 along with rotation of the sheath 108. The balloon 104 can be deflated by draining the fluid back through the proximal end 116 of the shaft 106. A basket apparatus 102 can be deployed from the distal end 110 of a shaft 106 via a basket actuator 118. The basket actuator 118 can be accessed by the professional near the proximal end 116 of the shaft 106. Alternatively, the basket apparatus 102 may be deployed by manual movement of a rod in a shaft lumen near the proximal end 116 of the shaft 106. A professional can manually move a proximal end of the rod distally relative to the shaft, thereby deflecting the basket 102 out the distal end 110 of the shaft 106 and moving the basket 102 to the deployed position. In a number of examples, the basket 102 may alternatively be initially deployed and fixed at the distal end of the shaft. Where the basket is initially deployed, the professional may manipulate the deployed basket 102 without the need for actuation upon the basket 102. A secondary instrument 136 can be inserted into the renal pelvis 134 through the port defined by the sheath 108. The secondary instrument 136 can be capable of performing lithotripsy, in which the calculus is reduced in size by the application of sound energy, laser energy, electrohydraulic energy, or other outside source of energy to fragment the calculus and reduce the size of the target body. As depicted in FIG. 7, a secondary instrument 136, such as a laser fiber or lithotripter, can be utilized to fragment a calculus 130 and to ease extraction. The shaft 106 which can be attached to the deflated balloon 104 can be manipulated within the port defined by the sheath 108 at the proximal end 116 to capture a calculus 130, or calculus fragments, or both.

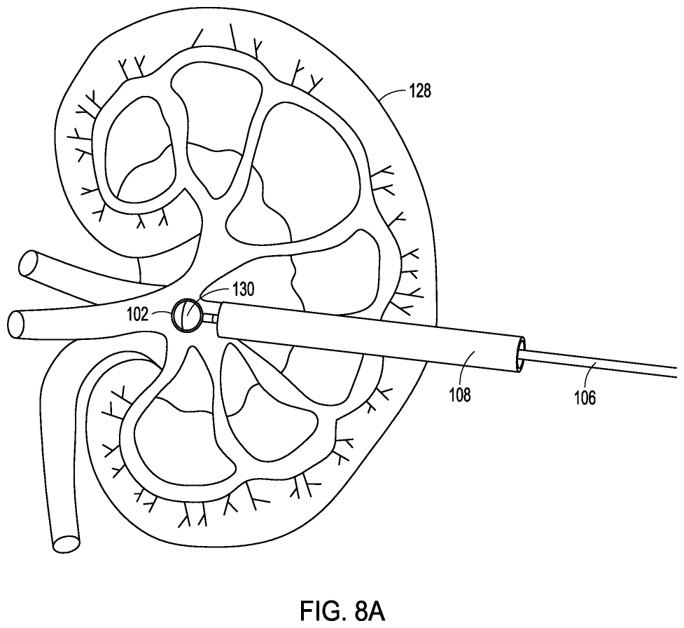

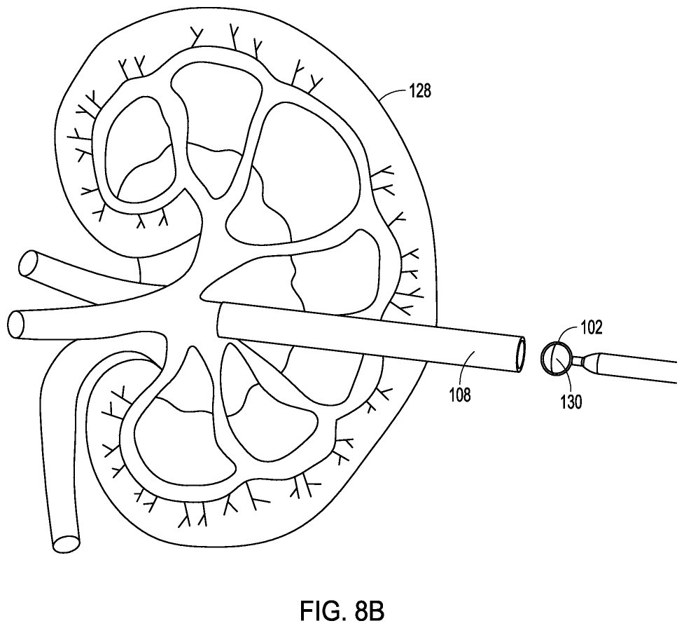

FIGS. 8A and 8B show an example of a device in use. In FIG. 8A, a calculus 130 is shown captured in the basket 102. The basket 102 and captured contents can be retrieved through the port defined by the sheath 108, as depicted in FIG. 8B. After the captured contents have been removed from the patient and released from the basket 102, the basket 102 can be re-inserted one or more times into the patient's anatomy to remove all or most of any remaining calculi or calculus fragments. If the size of the captured contents exceeds a predetermined size limit, the device can be configured such as to not allow the basket 102 to be retrieved through the sheath 108. A releasing mechanism can be initiated such as to allow the captured contents to be released. A professional can alternate between lithotripsy and use of the PCNL device of the present disclosure to perform a procedure.

The above description includes references to the accompanying drawings, which form a part of the detailed description. The drawings show, by way of illustration, specific examples in which the invention can be practiced. These examples are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein. In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A combination access and removal device for use in percutaneous nephrolithotomy to remove a kidney stone from a patient, the device comprising:
an inflatable balloon configured to dilate a percutaneous nephrolithotomy access route from an external location on the patient to a location within a kidney of the patient;
an elongated shaft extending through a central lumen defined by the balloon and attached thereto;
a basket apparatus deployable via a distal end of the elongated shaft and configured to capture a kidney stone or fragment or both; and
a sheath configured to be extended over the inflatable balloon to provide a port extending along the percutaneous nephrolithotomy access route to provide secondary instrument access to the kidney including when the shaft is located within the port with the balloon in a deflated state;
wherein the basket is configured to be retrieved through the port via retraction.

2. The device of claim 1, wherein the device is configured to allow for entry of the basket through a distal end of the port without removal of the balloon.

3. The device of claim 1, wherein the basket is configured to be collapsible within the distal end of the shaft.

4. The device of claim 1, further comprising a basket actuator located at a proximal end of the shaft, the basket actuator being configured to deploy the basket apparatus when the shaft is located within the port.

5. The device of claim 1, wherein the basket is configured be capable of withstanding lithotripsy via the secondary instrument and remain safely removable.

6. The device of claim 5, wherein the basket is configured to break non-traumatically when struck by the secondary instrument.

7. The device of claim 1, wherein the balloon has a length of at least 12 centimeters.

8. The device of claim 1, wherein the balloon is no longer than 14 centimeters.

9. The device of claim 1, wherein the balloon in an inflated state has an outer diameter of at least 10 millimeters.

10. The device of claim 1, wherein the balloon in an inflated state has an outer diameter of no greater than 12 millimeters.

11. The device of claim 1, wherein the shaft includes a longitudinal lumen and further comprising a basket manipulation member configured to extend through the lumen of the shaft and connecting to the basket at a distal end of the basket manipulation member, to permit removal of a kidney stone, fragment, or both via the lumen of the shaft.

12. The device of claim 11, wherein the basket manipulation member is configured to provide:
a first position wherein the basket is near the distal end of the shaft; and
a second position wherein the basket extends outward from the distal end of the shaft.

13. The device of claim 12, further comprising an extension actuator located at a proximal end of the shaft, the extension actuator being configured to extend the basket manipulation member from the first position to the second position.

14. A combination access and removal device for use in percutaneous nephrolithotomy to remove a kidney stone from a patient, the device comprising:
an inflatable balloon configured to dilate a percutaneous nephrolithotomy access route from an external location on the patient to a location within a kidney of the patient, wherein the balloon is configured to be inflated with a non-gaseous medium;
an elongated shaft extending through a central lumen defined by the balloon and attached thereto;
a sheath configured to be extended over the inflatable balloon to provide a port extending along the percutaneous nephrolithotomy access route to provide secondary instrument access to the kidney including when the shaft is located within the port with the balloon in a deflated state; and
a basket apparatus deployable via a distal end of the shaft on which the balloon resides and configured to capture a kidney stone or fragment or both;
wherein the basket apparatus is configured to allow for deployment and retrieval of the basket through a distal end of the port without removal of the balloon; and

15. A method for performing percutaneous nephrolithotomy to remove a kidney stone from a patient, the method comprising:
dilating, using an inflatable balloon on an elongated shaft, a percutaneous nephrolithotomy access route from an external location on the patient to a location within a kidney of the patient;
providing a port, by extending a sheath over the inflatable balloon, along the percutaneous nephrolithotomy access route from the external location on the patient to the location within the kidney of the patient; and
capturing, using a basket deployed via a distal end of the shaft, a kidney stone or fragment, or both.

16. The method of claim 15, comprising retrieving a captured stone or fragment or both by retracting the basket through the port.

17. The method of claim 16, comprising deflating the balloon when the port has been provided.

18. The method of claim 17, wherein deflating the balloon permits concurrent access of the shaft and a secondary instrument to the port.

19. The method of claim 18, comprising manipulating the basket toward a therapy location using the secondary instrument.

20. The method of claim 15, wherein dilating comprises inflating the balloon with a non-gaseous medium to a pressure of at least 18 atmospheres.
